(19) United States
(12) Patent Application Publication
(10) Pub. No.: US 2024/0130672 A1
(43) Pub. Date: Apr. 25, 2024

(54) Headache Detection and Treatment System

(71) Applicant: Neurostim Technologies LLC, Wellesley, MA (US)

(72) Inventors: Constance CARDASIS, Jupiter, FL (US); William C. ALTMANN, Austin, TX (US); Ann G. YEE, Brookline, MA (US); Pheng Dang, San Jose, CA (US); Hoo-min D. TOONG, Cambridge, MA (US)

(21) Appl. No.: 18/487,503

(22) Filed: Oct. 15, 2023

Related U.S. Application Data
(60) Provisional application No. 63/380,303, filed on Oct. 20, 2022.

(51) Int. Cl. A61B 5/00; A61B 5/01; A61N 1/36

(52) U.S. Cl. CPC A61B 5/4836; A61B 5/01; A61B 5/4076; A61B 5/6814; A61B 5/6833; A61N 1/36021; A61N 1/36025; A61N 1/36031; A61N 1/36034; A61B 2560/0242; A61B 2560/0468; A61B 2562/0204; A61B 2562/0219; A61B 2562/0223; A61B 2562/0271

(57) ABSTRACT

Example inventions treat a headache by affixing a patch externally on a dermis of a user on a head of the user adjacent to one or more targeted nerves, the patch comprising a flexible substrate, a processor directly coupled to the substrate, and electrodes directly coupled to the substrate. Example inventions activate the patch to initiate a treatment session, the activating comprising generating electrical stimuli to the targeted nerves via the electrodes.

### CROSS REFERENCE TO RELATED APPLICATIONS

This application claims priority to U.S. Provisional Patent Application Ser. No. 63/380,303, filed on Oct. 20, 2022, the disclosure of which is hereby incorporated by reference.

### FIELD

Example inventions are directed to systems and methods for treating headaches by stimulating the nerves with transcutaneous electrical stimulation.

### BACKGROUND INFORMATION

A headache is a common medical condition characterized by pain or discomfort in the head or upper neck region. Headaches can vary in intensity, location, and duration, and they may be caused by a wide range of factors, including tension, stress, dehydration, sinus problems, eyestrain, or underlying medical conditions. Headaches can be categorized into different types, such as tension headaches, migraines, cluster headaches, and more, based on their specific characteristics and causes. The pain associated with a headache can range from mild to severe and may be accompanied by other symptoms like sensitivity to light and noise, nausea, or vomiting. Treatment for headaches often depends on their underlying cause and can include rest, hydration, over-the-counter pain relievers, or prescription medications as recommended by a healthcare professional.

### BRIEF DESCRIPTION OF THE DRAWINGS

FIG. 1 illustrates an example patch that is affixed to a location behind an ankle bone of a user in example inventions.

FIG. 2 is a block diagram illustrating hardware/software related elements of an example of the patch of FIG. 1.

FIG. 3A is a circuit diagram of an example of a boosted voltage circuit that provides feedback.

FIG. 3B is a circuit diagram of an example of a charge application circuit that uses an output of the boosted voltage circuit.

FIG. 4 is a flow diagram of the functionality of the controller of monitoring and controlling the output voltage, including its ramp rate.

FIG. 5 is a flow diagram in accordance with one example of an adaptive protocol.

FIG. 6 is a Differential Integrator Circuit used in the adaptive protocol in accordance with one example.

FIG. 7 is a table relating charge duration vs. frequency to provide feedback to the adaptive protocol in accordance with one example.

FIG. 8 is an illustration of components of a headache detection and treatment system in accordance with example inventions.

FIG. 9 illustrates the nerves of the scalp.

FIG. 10A illustrates Frontal Patch applied to the User on the forehead over one or more of the bilateral supraorbital or supratrochlear nerves in accordance with example inventions.

FIG. 10B illustrates Dorsal Patch applied to the User on the back of the head over one or more of the bilateral Occipital Nerves in accordance with example inventions.

FIG. 11A illustrates the neurological structures of the head related to headache.

FIG. 11B illustrates the connection from the Occipital Nerves to the spinal column

FIG. 12 illustrates example stimulation waveforms for treating headaches in accordance with example inventions.

### DETAILED DESCRIPTION

A non-invasive nerve/tissue patch/activator in accordance with various examples disclosed herein includes novel circuitry to adequately boost voltage to a required level and to maintain a substantially constant level of charge for nerve activation or tissue stimulation. Optionally, a feedback loop provides for an automatic determination and adaptation of the applied charge level. In example inventions, the patch is used facilitate the detection and treatment of headaches with or without the use of medications. An integrated system is placed on the skin of a user and can be activated, adjusted and used with or without the help of a medical professional. The integrated system includes hardware and software to monitor biometrics related to headache treatment, and to stimulate the skin to continue the treatment, while also providing a closed-loop system. In contrast, known solutions of subdural electrical treatment for headaches involves significant expense, and risks related to surgical procedures and the implanted device.

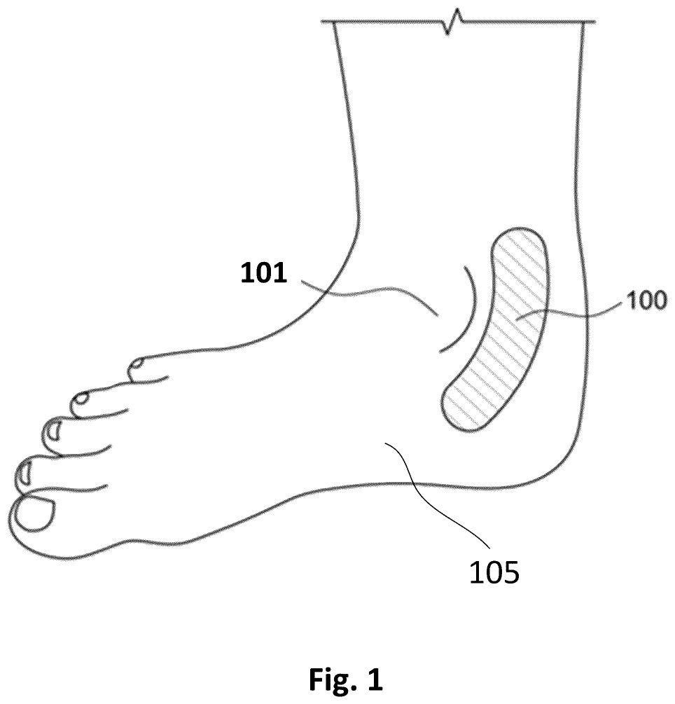

FIG. 1 illustrates an example patch 100, also referred to as a smart band aid or smartpad or Topical Nerve/Tissue Activator (“TNA”) or topical nerve activation and tissue stimulation patch, that is affixed to a location behind an ankle bone 101 of a user 105 in one example use, but as disclosed below is affixed to the head area when used for detecting and treating headaches.

Patch 100 is used to stimulate nerves and tissues and is convenient, unobtrusive, self-powered, and may be controlled from a smartphone or other control device. This has the advantage of being non-invasive, controlled by consumers themselves, and potentially distributed over the counter without a prescription. Patch 100 provides a means of stimulating nerves and tissues without penetrating the dermis, and can be applied to the surface of the dermis at a location appropriate for detecting and treating headaches. In examples, patch 100 is applied by the user and is disposable.

Patch 100 in examples can be any type of device that can be fixedly attached to a user, using adhesive in some examples, and includes a processor/controller and instructions that are executed by the processor, or a hardware implementation without software instructions, as well as electrodes that apply an electrical stimulation to the surface of the user's skin, and associated electrical circuitry. Patch 100 in one example provides topical nerve or tissue activation/stimulation on the user to provide benefits to the user, including bladder management for an overactive bladder (“OAB”) or headache detection and treatment.

Patch 100 in one example can include a flexible substrate, a malleable dermis conforming bottom surface of the substrate including adhesive and adapted to contact the dermis, a flexible top outer surface of the substrate approximately parallel to the bottom surface, a plurality of electrodes positioned on the patch proximal to the bottom surface and located beneath the top outer surface and directly contacting the flexible substrate, electronic circuitry (as disclosed herein) embedded in the patch and located beneath the top outer surface and integrated as a system on a chip that is directly contacting the flexible substrate, the electronic circuitry integrated as a system on a chip or discrete components and including an electrical signal generator integral to the malleable dermis conforming bottom surface configured to electrically activate the one or more electrodes, a signal activator coupled to the electrical signal generator, a nerve/tissue stimulation sensor that provides feedback in response to a stimulation of one or more nerves or tissues, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to stimulate one or more nerves or tissues of a user wearing patch 100 at least at one location proximate to patch 100. Additional details of examples of patch 100 beyond the novel details disclosed herein are disclosed in U.S. Pat. No. 10,016,600, entitled “Topical Neurological Stimulation”, the disclosure of which is hereby incorporated by reference.

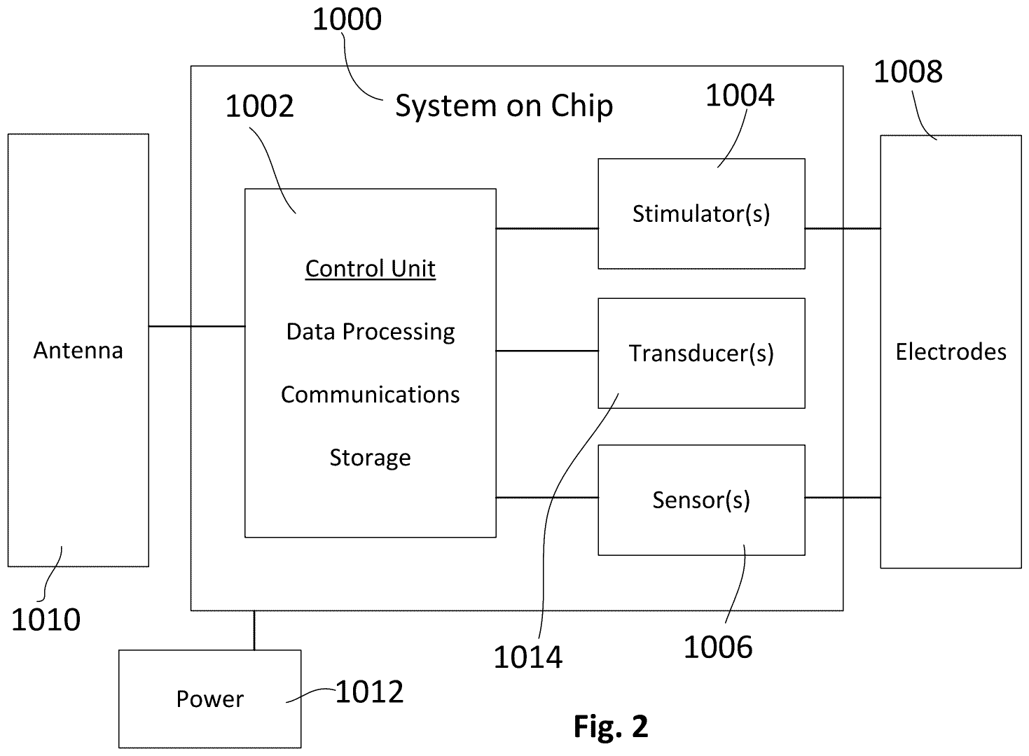

FIG. 2 is a block diagram illustrating hardware/software related elements of an example of patch 100 of FIG. 1. Patch 100 includes electronic circuits or chips 1000 that perform the functions of: communications with an external control device, such as a smartphone or fob, or external processing such as cloud based processing devices, nerve and tissue activation via electrodes 1008 that produce a wide range of electric fields according to a treatment regimen, and a wide range of sensors 1006 such as, but not limited to, mechanical motion and pressure, temperature, humidity, acoustic, chemical and positioning sensors. In another example, patch 100 includes transducers 1014 to transmit signals to the tissue or to receive signals from the tissue.

One arrangement is to integrate a wide variety of these functions into a system on a chip 1000. Within this is shown a control unit 1002 for data processing, communications, transducer interface and storage, and one or more stimulators 1004 and sensors 1006 that are connected to electrodes 1008. Control unit 1002 can be implemented by a general purpose processor/controller, or a specific purpose processor/controller, or a special purpose logical circuit. An antenna 1010 is incorporated for external communications by control unit 1002. Also included is an internal power supply 1012, which may be, for example, a battery. Other examples may include an external power supply. It may be necessary to include more than one chip to accommodate a wide range of voltages for data processing and stimulation. Electronic circuits and chips will communicate with each other via conductive tracks within the device capable of transferring data and/or power.

Patch 100 interprets a data stream from control unit 1002 to separate out message headers and delimiters from control instructions. In one example, control instructions include information such as voltage level and pulse pattern. Patch 100 activates stimulator 1004 to generate a stimulation signal to electrodes 1008 placed on the tissue according to the control instructions. In another example, patch 100 activates transducer 1014 to send a signal to the tissue. In another example, control instructions cause information such as voltage level and a pulse pattern to be retrieved from a library stored by patch 100, such as storage within control unit 1002.

Patch 100 receives sensory signals from the tissue and translates them to a data stream that is recognized by control unit 1002. Sensory signals can include electrical, mechanical, acoustic, optical and chemical signals. Sensory signals are received by patch 100 through electrodes 1008 or from other inputs originating from mechanical, acoustic, optical, or chemical transducers. For example, an electrical signal from the tissue is introduced to patch 100 through electrodes 1008, is converted from an analog signal to a digital signal and then inserted into a data stream that is sent through antenna 1010 to the external control device. In another example, an acoustic signal is received by transducer 1014, converted from an analog signal to a digital signal and then inserted into a data stream that is sent through the antenna 1010 to the external control device. In another example a pH sensor will detect the user's skin pH or the pH of a formulation included with the patch and sends that data to the control unit. In some examples, sensory signals from the tissue are directly interfaced to the external control device for processing.

In examples of patch 100 disclosed above, when being used for therapeutic treatment such as bladder management for OAB or headache treatment, there is a need to control the voltage by boosting the voltage to a selected level and providing the same level of charge upon activation to a mammalian nerve or tissue. Further, there is a need to conserve battery life by selectively using battery power. Further, there is a need to create a compact electronics package to facilitate mounting the electronics package on a relatively small mammalian dermal patch in the range of the size of an ordinary band aid.

### Adaptive Circuit

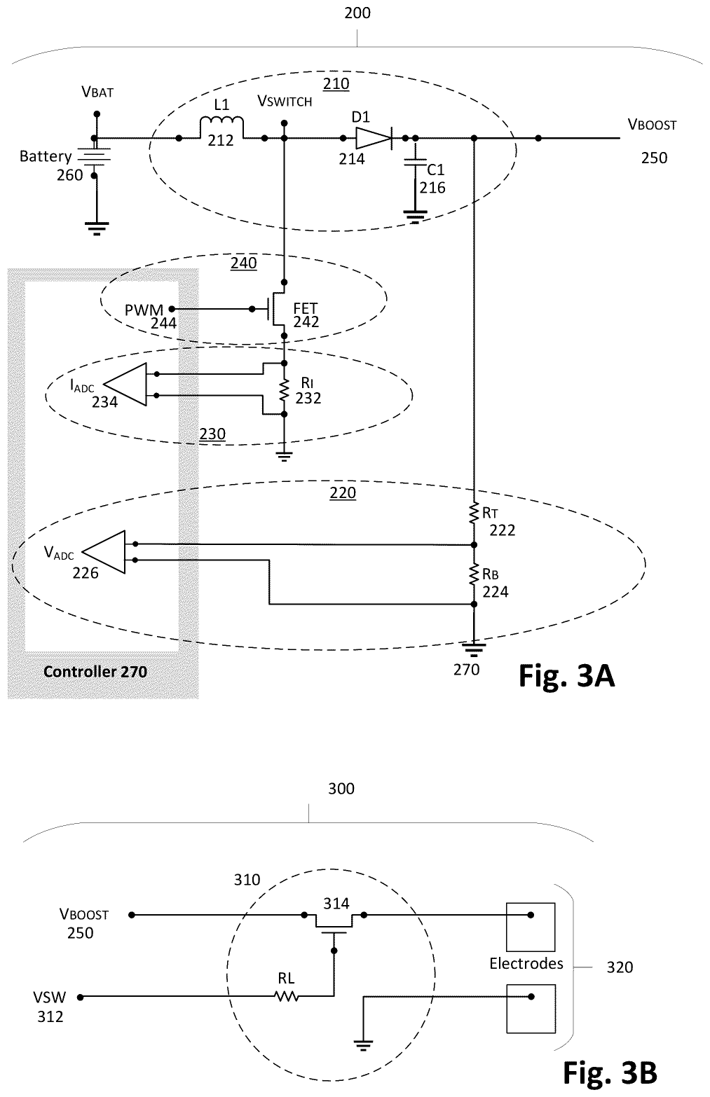

To meet the above needs, examples implement a novel boosted voltage circuit that includes a feedback circuit and a charge application circuit. FIG. 3A is a circuit diagram of an example of the boosted voltage circuit 200 that provides feedback. FIG. 3B is a circuit diagram of an example of a charge application circuit 300 that uses an output of boosted voltage circuit 200. Boosted voltage circuit 200 includes both electrical components and a controller/processor 270 that includes a sequence of instructions that together modify the voltage level of activation/stimulation delivered to the external dermis of user 105 by patch 100 through electrodes. Controller/processor 270 in examples implements control unit 1002 of FIG. 2.

Boosted voltage circuit 200 can replace an independent analog-controlled boost regulator by using a digital control loop to create a regulated voltage, output voltage 250, from the battery source. Output voltage 250 is provided as an input voltage to charge application circuit 300. In examples, this voltage provides nerve stimulation currents through the dermis/skin to treat headaches. Output voltage 250, or “V_{Boost}”, at voltage output node 250, uses two digital feedback paths 220, 230, through controller 270. In each of these paths, controller 270 uses sequences of instructions to interpret the measured voltages at voltage monitor 226, or “V_{ADC}” and current monitor 234, or “I_{ADC}”, and determines the proper output control for accurate and stable output voltage 250.

Boosted voltage circuit 200 includes an inductor 212, a diode 214, a capacitor 216 that together implement a boosted converter circuit 210. A voltage monitoring circuit 220 includes a resistor divider formed by a top resistor 222, or “R_{T}”, a bottom resistor 224, or “R_{B}” and voltage monitor 226. A current monitoring circuit 230 includes a current measuring resistor 232, or “R_{I}” and current monitor 234. A pulse width modulation (“PWM”) circuit 240 includes a field-effect transistor (“FET”) switch 242, and a PWM driver 244. Output voltage 250 functions as a sink for the electrical energy. An input voltage 260, or “V_{BAT}”, is the source for the electrical energy, and can be implemented by power 1012 of FIG. 2.

PWM circuit 240 alters the “on” time within a digital square wave, fixed or variable frequency signal to change the ratio of time that a power switch is commanded to be “on” versus “off.” In boosted voltage circuit 200, PWM driver 244 drives FET switch 242 to “on” and “off” states.

In operation, when FET switch 242 is on, i.e., conducting, the drain of FET switch 242 is brought down to Ground/GND or ground node 270. FET switch 242 remains on until its current reaches a level selected by controller 270 acting as a servo controller. This current is measured as a representative voltage on current measuring resistor 232 detected by current monitor 234. Due to the inductance of inductor 212, energy is stored in the magnetic field within inductor 212. The current flows through current measuring resistor 232 to ground until FET switch 242 is opened by PWM driver 244.

When the intended pulse width duration is achieved, controller 270 turns off FET switch 242. The current in inductor 212 reroutes from FET switch 242 to diode 214, causing diode 214 to forward current. Diode 214 charges capacitor 216. Therefore, controller 270 controls the voltage level at capacitor 216.

Output voltage 250 is controlled using an outer servo loop of voltage monitor 226 and controller 270. Output voltage 250 is measured by the resistor divider using top resistor 222, bottom resistor 224, and voltage monitor 226. The values of top resistor 222 and bottom resistor 224 are selected to keep the voltage across bottom resistor 224 within the monitoring range of voltage monitor 226. Controller 270 monitors the output value from voltage monitor 226.

Charge application circuit 300 includes a pulse application circuit 310 that includes an enable switch 314. Controller 270 does not allow enable switch 314 to turn on unless output voltage 250 is within a desired upper and lower range of the desired value of output voltage 250. Pulse application circuit 310 is operated by controller 270 by asserting an enable signal 312, or “VSW”, which turns on enable switch 314 to pass the electrical energy represented by output voltage 250 through electrodes 320. At the same time, controller 270 continues to monitor output voltage 250 and controls PWM driver 244 to switch FET switch 242 on and off and to maintain capacitor 216 to the desired value of output voltage 250.

The stability of output voltage 250 can be increased by an optional inner feedback loop through FET Switch 242, current measuring resistor 232, and current monitor 234. Controller 270 monitors the output value from current monitor 234 at a faster rate than the monitoring on voltage monitor 226 so that the variations in the voltages achieved at the cathode of diode 214 are minimized, thereby improving control of the voltage swing and load sensitivity of output voltage 250.

In one example, a voltage doubler circuit is added to boosted voltage circuit 200 to double the high voltage output or to reduce voltage stress on FET 242. The voltage doubler circuit builds charge in a transfer capacitor when FET 242 is turned on and adds voltage to the output of boosted voltage circuit 200 when FET 242 is turned off.

As described, in examples, controller 270 uses multiple feedback loops to adjust the duty cycle of PWM driver 244 to create a stable output voltage 250 across a range of values. Controller 270 uses multiple feedback loops and monitoring circuit parameters to control output voltage 250 and to evaluate a proper function of the hardware. Controller 270 acts on the feedback and monitoring values in order to provide improved patient safety and reduced electrical hazard by disabling incorrect electrical functions.

In some examples, controller 270 implements the monitoring instructions in firmware or software code. In some examples, controller 270 implements the monitoring instructions in a hardware state machine.

In some examples, voltage monitor 226 is an internal feature of controller 270. In some examples, voltage monitor 226 is an external component, which delivers its digital output value to a digital input port of controller 270.

In some examples, current monitor 234 is an internal feature of controller 270. In some examples, current monitor 234 is an external component, which delivers its digital output value to a digital input port of controller 270.

An advantage of boosted voltage circuit 200 over known circuits is decreased component count which may result in reduced costs, reduced circuit board size and higher reliability. Further, boosted voltage circuit 200 provides for centralized processing of all feedback data which leads to faster response to malfunctions. Further, boosted voltage circuit 200 controls outflow current from V_{BAT} 260, which increases the battery's lifetime and reliability.

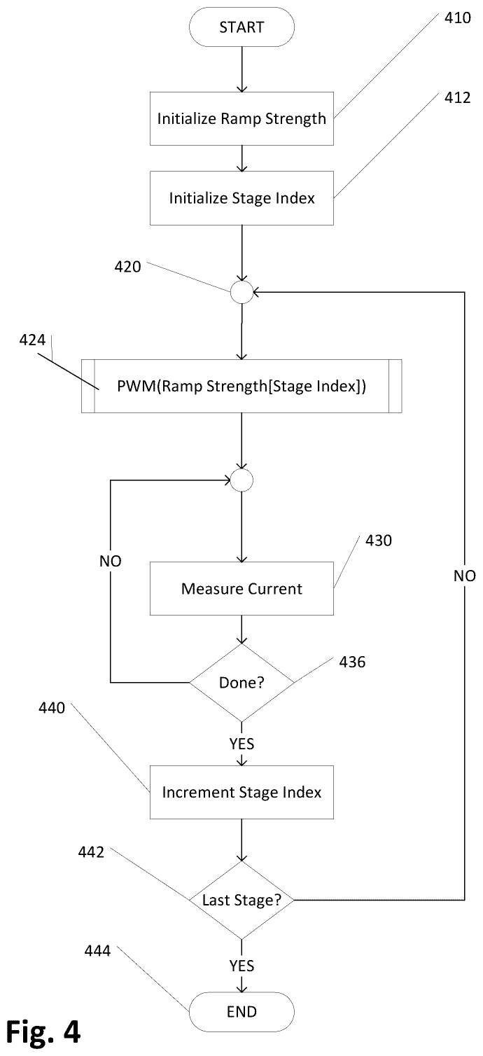

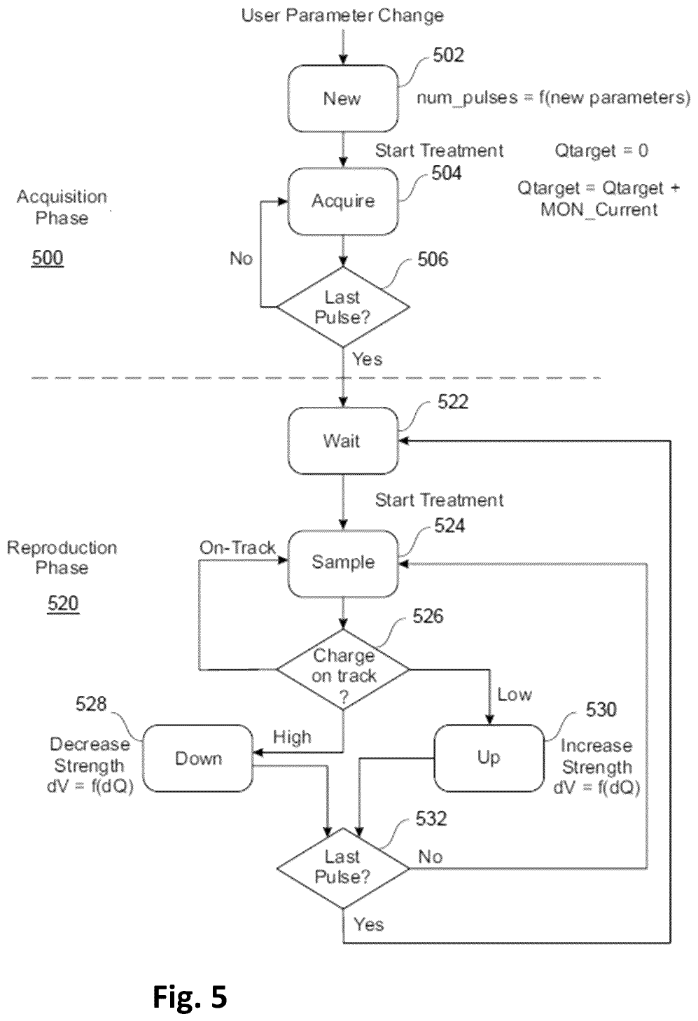

FIG. 4 is a flow diagram of the functionality of controller 270 of monitoring and controlling output voltage 250, including its ramp rate. In one example, the functionality of the flow diagram of FIG. 4, and FIG. 5 below, is implemented by software stored in memory or other computer readable or tangible medium, and executed by a processor. In other examples, the functionality may be performed by hardware (e.g., through the use of an application-specific integrated circuit (“ASIC”), a programmable gate array (“PGA”), a field programmable gate array (“FPGA”), etc.), or any combination of hardware and software.

The pulse width modulation of FET switch 242 is controlled by one or more pulses for which the setting of each pulse width allows more or less charge to accumulate as a voltage at capacitor 216 through diode 214. This pulse width setting is referred to as the ramp strength and it is initialized at 410. Controller 270 enables each pulse group in sequence with a pre-determined pulse width, one stage at a time, using a stage index that is initialized at 412. The desired ramp strength is converted to a pulse width at 424, which enables and disables FET switch 242 according to the pulse width. During the intervals when FET switch 242 is “on”, the current is measured by current monitor 234 at 430 and checked against the expected value at 436. When the current reaches the expected value, the stage is complete and the stage index is incremented at 440. If the desired number of stages has been applied 442, then the functionality is complete. Otherwise, the functionality continues to the next stage at 420.

As a result of the functionality of FIG. 4, V_{BAT} 260 used in patch 100 operates for longer periods as the current drawn from the battery ramps at a low rate of increase to reduce the peak current needed to achieve the final voltage level 250 for each activation/stimulation treatment. PWM 244 duty cycle is adjusted by controller 270 to change the ramp strength at 410 to improve the useful life of the battery.

An open loop protocol to control current to electrodes in known neural stimulation devices does not have feedback controls. It commands a voltage to be set, but does not check the actual current delivered. A stimulation pulse is sent based on preset parameters and cannot be modified based on feedback from the patient's anatomy. When the device is removed and repositioned, the electrode placement varies. Also the humidity and temperature of the anatomy changes throughout the day. All these factors affect the actual charge delivery if the voltage is preset. Charge control is a patient safety feature and facilitates an improvement in patient comfort, treatment consistency and efficacy of treatment.

In contrast, examples of patch 100 includes features that address these shortcomings using controller 270 to regulate the charge applied by electrodes 320. Controller 270 samples the voltage of the stimulation waveform, providing feedback and impedance calculations for an adaptive protocol to modify the stimulation waveform in real time. The current delivered to the anatomy by the stimulation waveform is integrated using a differential integrator and sampled and then summed to determine the actual charge delivered to the user for a treatment, such as OAB or treating headaches. After every pulse in a stimulation event, this data is analyzed and used to modify, in real time, subsequent pulses.

This hardware adaptation allows a firmware protocol to implement the adaptive protocol. This protocol regulates the charge applied to the body by changing output voltage (“V_{BOOST}”) 250. A treatment is performed by a sequence of periodic pulses, which deliver charge into the body through electrodes 320. Some of the parameters of the treatment are fixed and some are user adjustable. The strength, duration and frequency may be user adjustable. The user may adjust these parameters as necessary for comfort and efficacy. The strength may be lowered if there is discomfort and raised if nothing is felt. The duration can be increased if the maximum acceptable strength results in an ineffective treatment.

### Adaptive Protocol

A flow diagram in accordance with one example of the adaptive protocol disclosed above is shown in FIG. 5. The adaptive protocol strives to repeatedly and reliably deliver a target charge (“Q_{target}”) in coulombs during a treatment and to account for any environmental changes. Therefore, the functionality of FIG. 5 is to adjust the charge level applied to a user based on feedback, rather than use a constant level. Environmental changes include changes to sensor signals received by the patch.

The mathematical expression of this protocol is as follows: Q_{target}=Q_{target} (A*dS+B*dT), where A is the Strength Coefficient—determined empirically, dS is the user change in Strength, B is the Duration Coefficient—determined empirically, and dT is the user change in Duration.

The adaptive protocol includes two phases in one example: Acquisition phase 500 and Reproduction phase 520. Any change in user parameters places the adaptive protocol in the Acquisition phase. When the first treatment is started, a new baseline charge is computed based on the new parameters. At a new acquisition phase at 502, all data from the previous charge application is discarded. In one example, 502 indicates the first time for the current usage where the user places patch 100 on a portion of the body and manually adjusts the charge level, which is a series of charge pulses, until it feels suitable, or any time the charge level is changed, either manually or automatically. The treatment then starts. The mathematical expression of this function of the application of a charge is as follows:

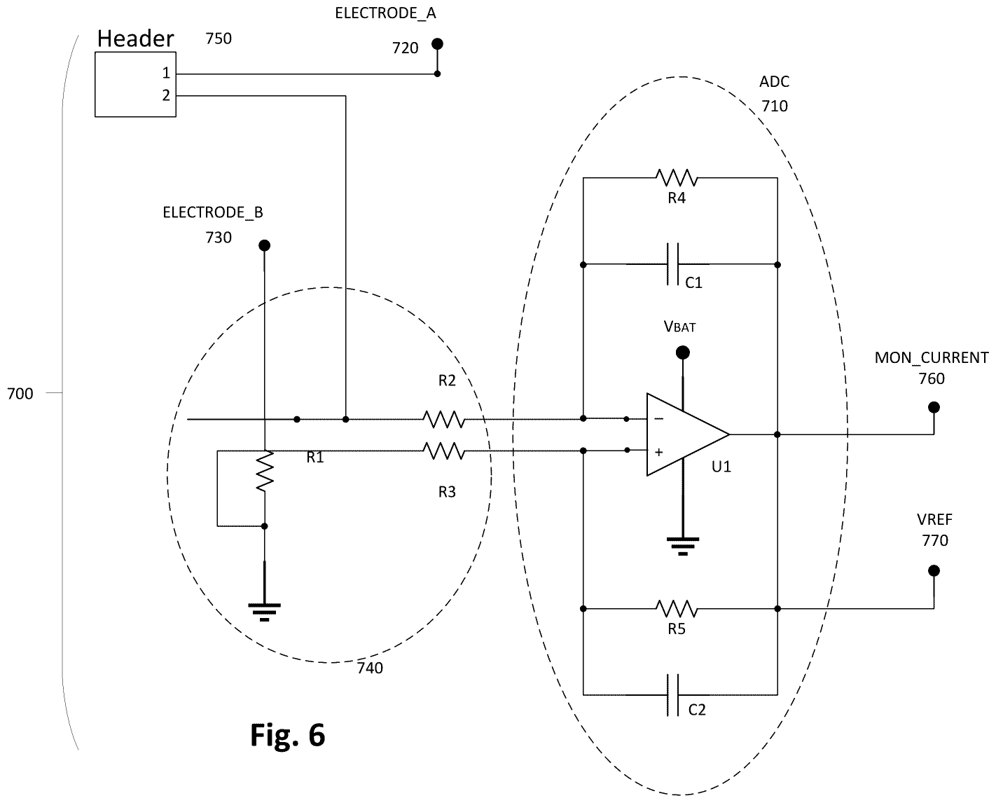

The charge delivered in a treatment is

$${Q}_{target} = {\sum }_{i = 1}^{T * f} {Q}_{pulse} ( i )$$

Where T is the duration; f is the count of pulses for one treatment (e.g., Hertz or cycles/second) of “Rep Rate”; Qₚᵤₗₛₑ (i) is the measured charge delivered by Pulse (i) in the treatment pulse train provided as a voltage MON_CURRENT that is the result of a Differential Integrator circuit shown in FIG. 6 (i.e., the average amount of charge per pulse). Differential Integrator circuit 700 of FIG. 6 is an example of a circuit used to integrate current measured over time and quantify the delivered charge and therefore determine the charge output over a treatment pulse. The number of pulses in the treatment is T*f.

As shown in of FIG. 6, MON_CURRENT 760 is the result of the Differential Integrator Circuit 700. The Analog to Digital Conversion (“ADC”) 710 feature is used to quantify voltage into a number representing the delivered charge. The voltage is measured between Electrode A 720 and Electrode B 730, using a Kelvin Connection 740. Electrode A 720 and Electrode B 730 are connected to a header 750. A reference voltage, VREF 770, is included to keep the measurement in range.

In some examples, Analog to Digital Conversion 710 is an internal feature of controller 270. In some examples, Analog to Digital Conversion 710 is an external component, which delivers its digital output value to a digital input port on Controller 270.

At 504 and 506, every pulse is sampled. In one example, the functionality of 504 and 506 lasts for 10 seconds with a pulse rate of 20 Hz. The result of Acquisition phase 500 is the target pulse charge of Q_{target}.

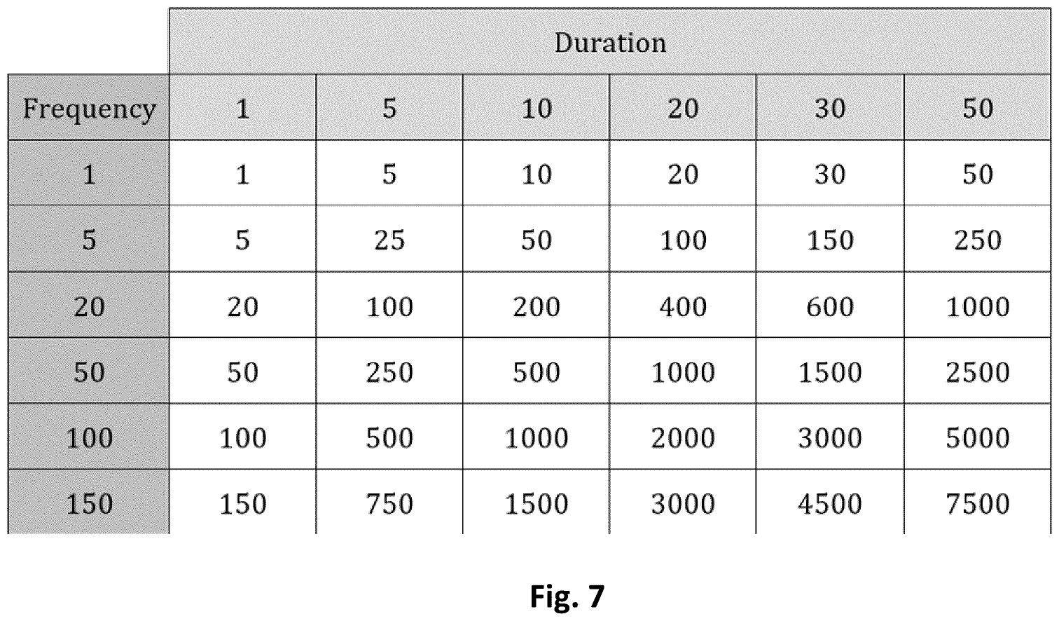

FIG. 7 is a table in accordance with one example showing the number of pulses per treatment measured against two parameters, frequency and duration. Frequency is shown on the Y-axis and duration on the X-axis. The adaptive protocol in general performs better when using more pulses. One example uses a minimum of 100 pulses to provide for solid convergence of charge data feedback, although a less number of pulses can be used in other examples. Referring to the FIG. 7, a frequency setting of 20 Hz and duration of 10 seconds produces 200 pulses. The range of duration of treatment extends to minutes, hours, and in some cases days, with the corresponding large number of pulses applied during that treatment time.

The reproduction phase 520 begins in one example when the user initiates another subsequent treatment after acquisition phase 500 and the resulting acquisition of the baseline charge, Q_{target}. For example, a full treatment cycle, as discussed above, may take 10 seconds. After, for example, a two-hour pause as shown at wait period 522, the user may then initiate another treatment. During this phase, the adaptive protocol attempts to deliver Q_{target} for each subsequent treatment. The functionality of reproduction phase 520 is needed because, during the wait period 522, conditions such as the impedance of the user's body due to sweat or air humidity may have changed. The differential integrator is sampled at the end of each Pulse in the Treatment. At that point, the next treatment is started and the differential integrator is sampled for each pulse at 524 for purposes of comparison to the acquisition phase Q_{target}. Sampling the pulse includes measuring the output of the pulse in terms of total electric charge. The output of the integrator of FIG. 6 in voltage, referred to as Mon_Current 760, is a direct linear relationship to the delivered charge and provides a reading of how much charge is leaving the device and entering the user. At 526, each single pulse is compared to the charge value determined in Acquisition phase 500 (i.e., the target charge) and the next pulse will be adjusted in the direction of the difference.

NUM_PULSES=(T*f)

After each pulse, the observed charge, Qₚᵤₗₛₑ(i), is compared to the expected charge per pulse.

Qₚᵤₗₛₑ(i)>Q_{target}/NUM_PULSES?

The output charge or “V_{BOOST}” is then modified at either 528 (decreasing) or 530 (increasing) for the subsequent pulse by:

dV(i)=G[Q_{target}/NUM_PULSES−Qₚᵤₗₛₑ(i)]

where G is the Voltage adjustment Coefficient—determined empirically. The process continues until the last pulse at 532.

A safety feature assures that the V_{BOOST} will never be adjusted higher by more than 10%. If more charge is necessary, then the repetition rate or duration can be increased.

In one example a boosted voltage circuit uses dedicated circuits to servo the boosted voltage. These circuits process voltage and/or current measurements to control the PWM duty cycle of the boosted voltage circuit's switch. The system controller can set the voltage by adjusting the gain of the feedback loop in the boosted voltage circuit. This is done with a digital potentiometer or other digital to analog circuit.

In one example, in general, the current is sampled for every pulse during acquisition phase 500 to establish target charge for reproduction. The voltage is then adjusted via a digital potentiometer, herein referred to as “Pot”, during reproduction phase 520 to achieve the established target_charge.

The digital Pot is calibrated with the actual voltage at startup. A table is generated with sampled voltage for each wiper value. Tables are also precomputed storing the Pot wiper increment needed for 1 v and 5 v output delta at each pot level. This enables quick reference for voltage adjustments during the reproduction phase. The tables may need periodic recalibration due to battery level.

In one example, during acquisition phase 500, the data set=100 pulses and every pulse is sampled and the average is used as the target_charge for reproduction phase 520. In general, fewer pulses provide a weaker data sample to use as a basis for reproduction phase 520.

In one example, during acquisition phase 500, the maximum data set=1000 pulses. The maximum is used to avoid overflow of 32bit integers in accumulating the sum of samples. Further, 1000 pulses in one example is a sufficiently large data set and collecting more is likely unnecessary.

After 1000 pulses for the above example, the target_charge is computed. Additional pulses beyond 1000 in the acquisition phase do not contribute to the computation of the target charge. In other examples, the maximum data set is greater than 1000 pulses when longer treatment cycle times are desired.

In one example, the first 3-4 pulses are generally higher than the rest so these are not used in acquisition phase 500. This is also accounted for in reproduction phase 520. Using these too high values can result in target charge being set too high and over stimulating on the subsequent treatments in reproduction phase 520. In other examples, more advanced averaging algorithms could be applied to eliminate high and low values.

In an example, there may be a safety concern about automatically increasing the voltage. For example, if there is poor connection between the device and the user's skin, the voltage may auto-adjust at 530 up to the max. The impedance may then be reduced, for example by the user pressing the device firmly, which may result in a sudden high current. Therefore, in one example, if the sample is 500 mv or more higher than the target, it immediately adjusts to the minimum voltage. This example then remains in reproduction phase 520 and should adjust back to the target current/charge level. In another example, the maximum voltage increase is set for a single treatment (e.g., 10V). More than that is not needed to achieve the established target_charge. In another example, a max is set for V_{BOOST} (e.g., 80V).

In various examples, it is desired to have stability during reproduction phase 520. In one example, this is accomplished by adjusting the voltage by steps. However, a relatively large step adjustment can result in oscillation or over stimulation. Therefore, voltage adjustments may be made in smaller steps. The step size may be based on both the delta between the target and sample current as well as on the actual V_{BOOST} voltage level. This facilitates a quick and stable/smooth convergence to the target charge and uses a more gradual adjustments at lower voltages for more sensitive users.

The following are the conditions that may be evaluated to determine the adjustment step.

delta-mon_current=abs(sample_mon_current−target_charge)

If delta_mon_current>500 mv and V_{BOOST}>20 V then step=5V for increase adjustments

(For decrease adjustments a 500 mv delta triggers emergency decrease to minimum Voltage)

If delta_mon_current>200 mv then step=1V

If delta_mon_current>100 mv and delta_mon_current>5%*sample_mon_current then step =1V

In other examples, new treatments are started with voltage lower than target voltage with a voltage buffer of approximately 10%. The impedance is unknown at the treatment start. These examples save the target_voltage in use at the end of a treatment. If the user has not adjusted the strength parameter manually, it starts a new treatment with saved target_voltage with the 10% buffer. This achieves target current quickly with the 10% buffer to avoid possible over stimulation in case impedance has been reduced. This also compensates for the first 3-4 pulses that are generally higher.

As disclosed, examples apply an initial charge level, and then automatically adjust based on feedback of the amount of current being applied. The charge amount can be varied up or down while being applied. Therefore, rather than setting and then applying a fixed voltage level throughout a treatment cycle, implementations of the invention measure the amount of charge that is being input to the user, and adjust accordingly throughout the treatment to maintain a target charge level that is suitable for the current environment.

The Adaptive Circuit described above provides the means to monitor the charge sent through the electrodes to the user's tissue and to adjust the strength and duration of sending charge so as to adapt to changes in the impedance through the electrode-to-skin interface and through the user's tissue such that the field strength at the target nerve or tissue is within the bounds needed to overcome the action potential of that nerve or tissue stimulation threshold at that location and activate a nerve impulse or tissue response. These changes in impedance may be caused by environmental changes, such as wetness or dryness of the skin or underlying tissue, or by applied lotion or the like; or by tissue changes, such as skin dryness; or by changes in the device's placement on the user's skin, such as by removing the patch and re-applying it in a different location or orientation relative to the target nerve or tissue; or by combinations of the above and other factors.

The combined circuits and circuit controls disclose herein generate a charge that is repeated on subsequent uses. The voltage boost conserves battery power by generating voltage on demand. The result is an effective and compact electronics package suitable for mounting on or in a fabric or similar material for adherence to a dermis that allows electrodes to be placed near selected nerves or tissues to be activated or stimulated.

### Headache Detection and Treatment

In some example inventions, patch 100, disclosed above, is used for detecting and treating and suppressing headaches. Example inventions provide an integrated system, including patch 100, which may be placed on the skin of the user on the user's head. The integrated system can be activated, adjusted and used with or without the help of a medical professional, and includes hardware and software to monitor biometrics related to headache treatment, and to stimulate the nerves to counteract headaches, with the additional ability to provide a closed-loop system.

Primary headaches, such as migraines, cluster and occipital neuralgias, afflict a significant portion of the population. The term “headache”, as used herein, encompass these, and other, types of headache physiologically related to the superior salivatory nucleus.

The pain associated with migraine and occipital headaches is thought to be the result of leakage of chemicals from nerves and dilated vessels in the scalp and meninges, such as calcitonin gene-related peptide (“CGRP”), and Substance P. These pain producing chemicals are sensed by vascular nociceptive fibers.

The superior salivatory nucleus has as inputs multiple cranial and cervical nerves, and as outputs similarly multiple pathways and nerves. Sensory nerves arrive, preganglionic parasympathetic motor nerves exit, and spinal and cranial nerve outputs go to the midbrain where pain sensation is processed and then experienced. The superior salivatory nucleus provides parasympathetic nerves supplying the vasculature and results in further dilatation of the scalp and meningeal vessels. Afferent nociceptor nerves on the blood vessels sense this dilation and send signals to the superior salivatory nucleus. These afferent signals work to trigger further activation signals in the parasympathetic efferent fibers which proceed from the superior salivatory nucleus to the blood vessels, creating a positive feedback loop which aggravates the headache with further dilation of the blood vessels.

In some headache modalities, the afferent nociceptor fibers are of the occipital nerves, which enter the trigeminal nucleus and send the information to the thalamus and cortex for the interpretation of pain. The trigeminal nucleus also has numerous afferent and efferent connections with other areas of the brain and brain stem, such as the hypothalamus and the superior salivatory nucleus. Some fibers go from the trigeminal nucleus to the superior salivatory nucleus and then as parasympathetic fibers to the sphenopalatine ganglion and then as post-ganglionic parasympathetic fibers to the blood vessels.

In some headache modalities, the afferent nociceptor fibers are of the auriculotemporal nerve. These enter the superior salivatory nucleus and continue on to the brain and brain stem. Other fibers go from the superior salivatory nucleus back to the blood vessels as parasympathetic fibers in the facial nerve.

In some headache modalities, the afferent nociceptor fibers are of the supraorbital nerve and the supratrochlear nerve. These branches of the frontal nerve proceed to the ophthalmic division of the trigeminal nerve.

The electrical stimulation of sensory afferent fibers of nerves going to the SSN relieves headache by means of one or both of regulation of efferent components described above, or modulation of pain in the higher central nervous system.

Transcutaneous electrical stimulation delivers a protocol to stimulate one or more of the afferent nerves or nerve branches which relate to the superior salivatory nucleus.

Transcutaneous electrical stimulation of one or more target nerves reduces headache pain by one or both of activating the target nerves such that their activation pulses cause an inhibitory effect on the nervous system's feedback loop which had been aggravating headache pain, or blocking activation of the target nerves to prevent activation pulses in that one or more nerves from passing toward the cell body and into the nervous system.

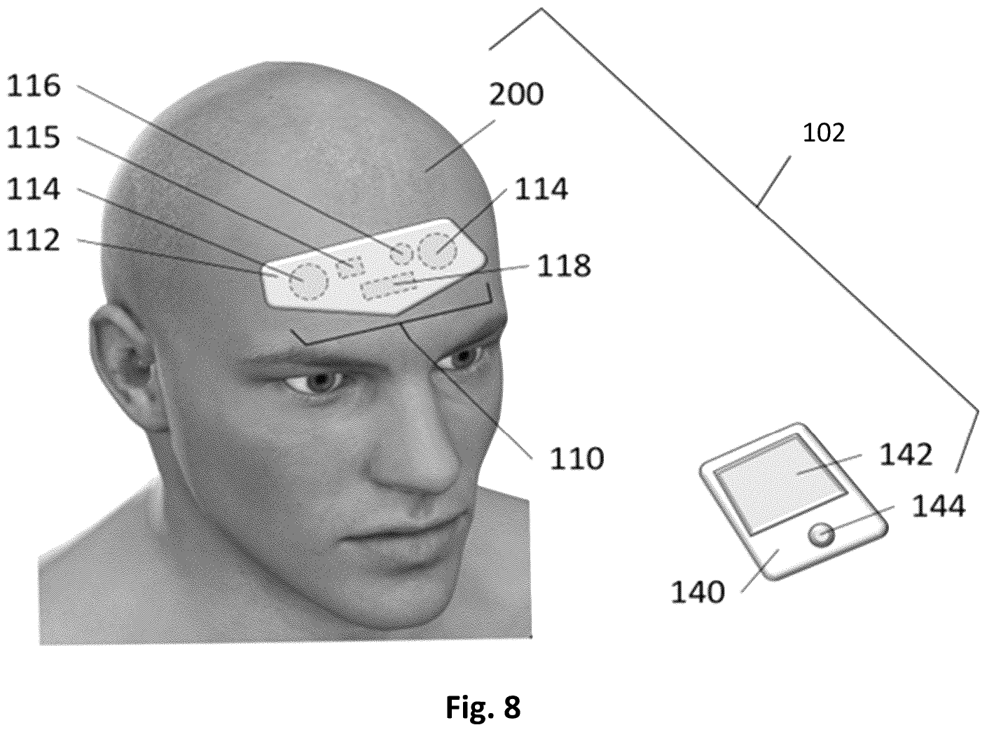

Example inventions electrical charge in a profile to stimulate one or more target nerves to treat headaches. FIG. 8 is an illustration of components of a headache detection and treatment system 102 in accordance with example inventions. System 102 includes a headache detection and treatment patch 110, which includes a securing mechanism 112 (e.g., adhesive layer), and one or more electrode pairs 114, with each pair having a positive electrode and a negative electrode (or multiple positive electrodes and a single negative electrode as disclosed below). Patch 110 further includes a power source 116, one or more sensors 115, a processor 118 and a flexible substrate. System 102 further includes an optional smart controller 140 (e.g., a smart phone), with a display 142, and an acknowledgment button 144. Patch 110 can be implemented by patch 100 previously described.

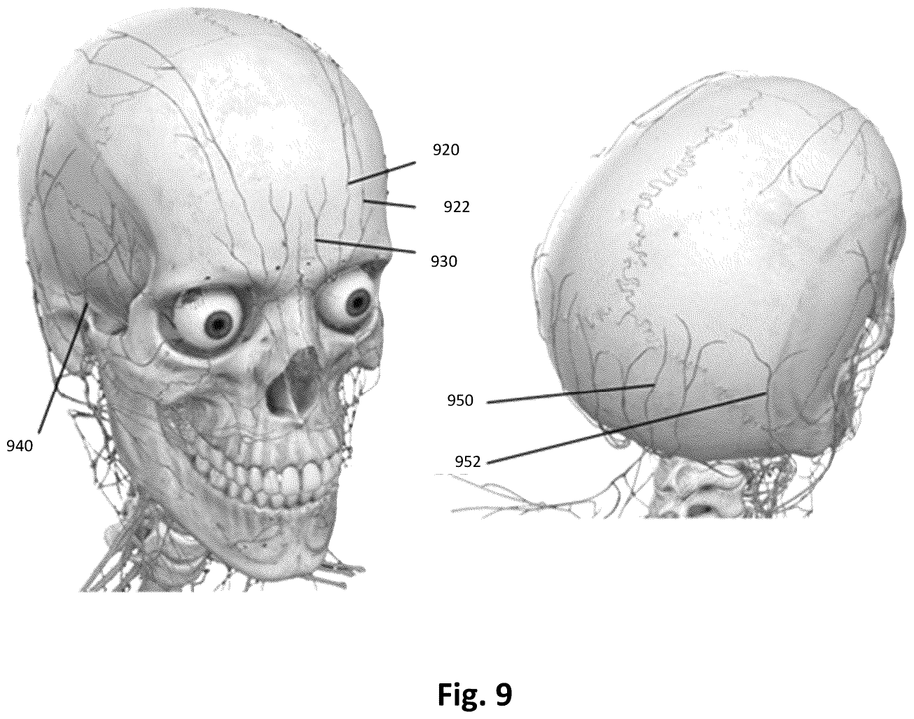

FIG. 9 illustrates the nerves of the scalp, with a Medial Branch of the Supraorbital Nerve 920, a Lateral Branch of the Supraorbital Nerve 922, a Supratrochlear Nerve 930, an Auriculotemporal Nerve 940, a Greater Occipital Nerve 950, and a Lesser Occipital Nerve 952. Each of these nerves is bilateral, having a Left and a Right instance.

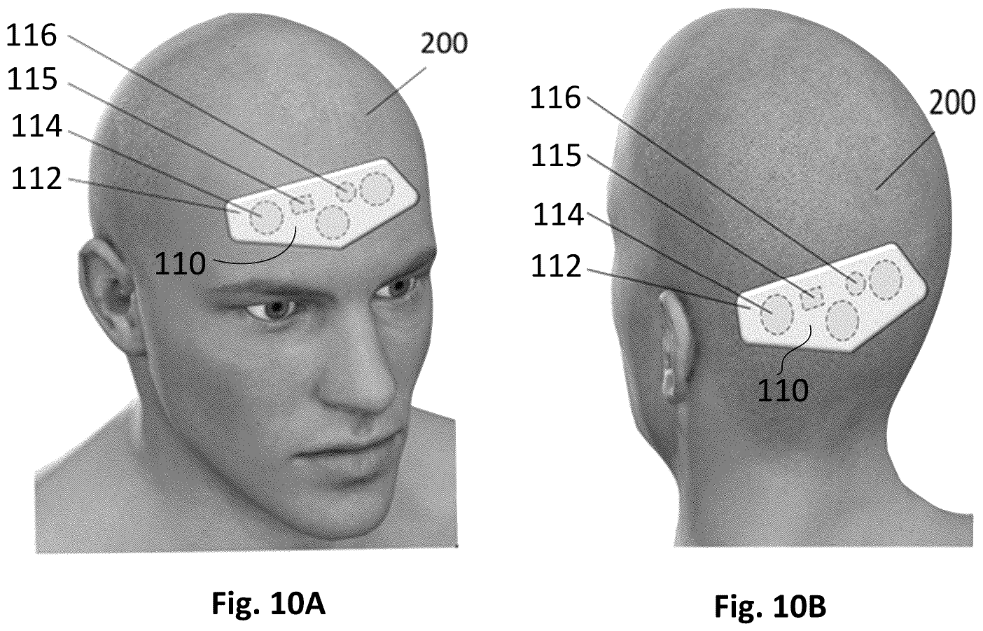

FIG. 10A illustrates Frontal Patch 110 applied to the User 200 on the forehead over one or more of the bilateral supraorbital or supratrochlear nerves in accordance with example inventions.

FIG. 10B illustrates Dorsal Patch 110 applied to the User 200 on the back of the head over one or more of the bilateral Occipital Nerves in accordance with example inventions.

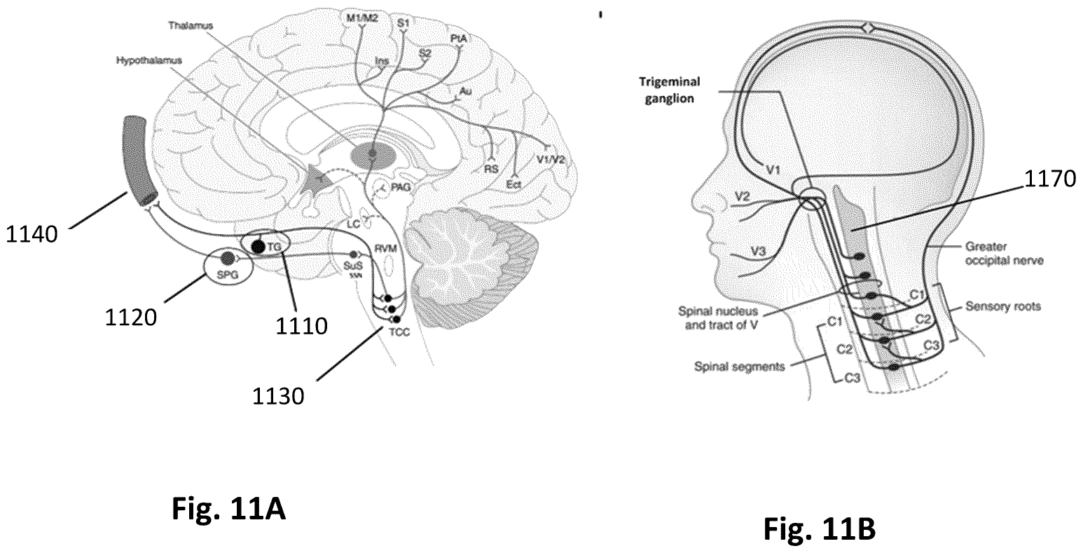

FIG. 11A illustrates the neurological structures of the head related to headache, with a Blood Vessel 1110, an Sphenopalatine Ganglion (SPG) 1120, a Trigeminal Nucleus (TG) 1130, a Superior Salivatory Nucleus (SSN) 1140, a Trigeminocervical Complex (TCC) 1150, a Hypothalamus 1160, and a Thalamus 1162.

FIG. 11B illustrates the connection from the Occipital Nerves to the spinal column and into the Trigeminal Caudalis Nucleus 1170 which connects to the SSN 1140.

Patch 110 is designed in a shape to conform to the skin when affixed to the skin and to be electronically effective at stimulating the tissues. Patch 110 may be used for headache monitoring as well as for delivering electrical stimulation to treat a detected headache. Patch 110 is electronically most effective for stimulation when the positive and negative electrodes are placed transversely across the path of the target nerve in contrast to axially along the path of the target nerve, which is not as electronically effective. The shape of patch 110 in examples is designed to minimize discomfort for the user 900 when affixed in the target location.

An example, patch 110 in FIG. 10A is affixed to the forehead, with a patch 110 on one or more of the left and right sides of the forehead, and the center front of the forehead. A sensor detects nerve activity in the ophthalmic nerves.

An example, patch 110 in FIG. 10B is affixed to the skin in an area extending from the C3 vertebra at the back of the neck to the occipital part of the skull. A sensor detects nerve activity in the occipital nerves.

An example, patch 110 in FIG. 10B is affixed to the skin in one or more of the C3, C4 and C5 dermatomes. An example, patch 110 is affixed to the skin at the temple over the Auriculotemporal Nerve 240, a common site of migraine pain.

An example, patch 110 is affixed to the skin on the side which is ipsilateral to the pain. An example, patch 110 is affixed to the skin bilaterally when the pain is sensed on both sides. An example, multiple patches 110 are affixed to the skin over each area where pain is sensed.

An example, patch 110 uses one electrode pair 114 to create a lateral electric field across the target nerve.

An example, patch 110 uses multiple positive electrodes and one or more negative electrodes to create a lateral electric field across one or more section of the target nerve, modifying the waveshapes or timings or both of the activation pulses from the multiple electrodes to direct the waveform energy at one or more specific points on the target nerve.

An example, patch 110 includes a layer of gel, such as hydrogel, between the electrode face of patch 110 and the surface of the skin. This gel layer improves the impedance matching between the TES Device and the skin.

The individual components of the Headache Detection and Treatment System 102 may be connected as peer devices in a Body Area Network, passing each other signals and sharing the tasks of data recording, real-time analysis, and closed-loop monitoring of the User 200.

Multiple Headache Detection and Treatment Systems 102, of different shapes or dimensions may be used in combination to detect and treat a headache. The selected Headache Detection and Treatment System may be adjusted during the course of treatment to adapt to the changing intensity of headache. These multiple Headache Detection and Treatment Systems may be connected as peer devices in a Body Area Network, with one or more controller.

### Sensing

An example, patch 110 includes one or more sensors 115, such as chemical, thermal, electrical field, magnetic field, audio, color and motion. Each sensor 115 is used to detect the degree of headache in a closed-loop system, combining the transcutaneous stimulation with sensing through sensors, and analysis of the sensor data through algorithms and/or machine learning, such that the stimulation protocol is adjusted in one or more of intensity, signal waveform, duration, to optimize efficacy and use of the electrical energy in patch 110.

An example, patch 110 includes one or more color Sensors 115. Patch 110 emits light onto the surface of the User's 200 skin. Patch 110 measures the light reflected back from one or both of the surface and subsurface of the skin, and calculates, by one or both of hardware and software, the color. The degree of pain is associated with change in color, such as the degree of redness. When the measured color differs from the color when the User has no headache pain in the target region, patch 110 deduces that there is pain in that target region.

An example, patch 110 includes one or more ultrasonic Sensor 115. Patch 110 emits ultrasonic energy, directed through the User's 200 skin. Patch 110 captures the ultrasonic return energy and calculates, by one of both of hardware and software, the size of blood vessels underlying Patch 110. The degree of pain is associated with the relative diameter of blood vessels. When the measured size of blood vessels exceeds the range measured when the User has no headache pain in that target region, Patch 110 deduces that there is pain at that target region.

An example, Patch 110 includes two or more thermal Sensors 115. Patch 110 uses one or more thermal sensors to read the ambient temperature at the site of Patch 110 affixed to the User's 200 skin. Patch 110′s use different one or more thermal Sensors to read the temperature on the skin in the region directly below the Patch 110. The degree of pain is associated with the skin temperature, relative to the ambient temperature, and when compared to the quiescent skin temperature when there is no pain. When the measured temperature exceeds the range measured when the User has no headache pain in that target region, Patch 110 deduces that there is pain at that target region.

An example, Patch 110 includes one or more reverse iontophoresis Sensors 115. Patch 110 emits an electric field, directed through the User's 200 skin. Patch 110 applies the electric field for a time sufficient to draw out through the skin a sample of CGRP or Substance P or other biochemical associated with pain. Patch 110 measures the amount of such drawn biochemical. The degree of pain is associated with the amount of such biochemicals in the body at the site of pain. When the measured amount of such biochemical exceeds the range measured when the User has no headache pain in that target region, Patch 110 deduces that there is pain at that target region.

An example, Patch 110 includes a chemical coating to the skin-facing surface of one or both of each Electrode Pair 114. The chemical coating is selected to change one or more of its electrical specifications when in the presence of a biochemical on the User's 200 skin surface. The biochemical may be CGRP, Substance P, or other biochemical associated with pain. Patch 110 measures the electrical performance of the chemical coating and compares the measurement to a baseline value which has been determined when the level of the selected biochemical is below that indicative of pain. Patch 110 determines that the User is experiencing pain when the electrical measurement exceeds the range when no pain is experienced.

An example, Patch 110 includes a chemical coating to the skin-facing surface of one or both of each Electrode Pair 114, separate from any coatings as described above. Patch 110 uses an electric field between the anode and cathode of each of the one or more Electrode Pairs to push the selected chemical molecules through the stratum corneum and into the dermis, in close contact with the underlying blood vessels. The chemical coating here is one of a set of antagonists, each of which when in the proximity of CGRP prevent the CGRP from binding to the blood vessels. By lowering the binding rate of CGRP, the selected chemical reduces the dilation effect from CGRP, and thereby reduces headache pain.

An example, when Patch 110 includes a chemical coating, the coating is applied to two or more Electrode Pairs 114, such that one Electrode Pair is used with its chemical coating until the chemical coating is depleted, at which time the Smart Controller 140 or Patch 110 stop using the first Electrode Pair and switch to using a second or subsequent Electrode Pair, this sequencing of Electrode Pair usage extending the usable life of Patch 110 on the User's 200 skin.

An example, Patch 110 establishes a baseline measurement using one or more Sensor 115 during a time when the individual User 200 has no headache. This baseline quantified value is used in comparison with values measured during the wearing of Patch 110 to detect a physiological change indicative of headache. The baseline measured value or values are stored in memory in one or both of Patch 110 and the Smart Controller 140.

An example, the baseline measurement is made whenever a Patch 110 is applied to the User's 200 skin. The baseline measurement is made when the User indicates, through the Smart Controller 140 or Patch 110, that there is no headache at that time.

An example, the baseline measurement is made when Patch 110 is applied to the skin, but not repeated when Patch 110 is replaced with a new Patch 110. The baseline measurement in this example is communicated from the Smart Controller 140 to the newly-applied Patch 110 to establish a baseline in the newly-attached Patch 110.

### User Interaction

An example, Patch 110 initiates a stimulation session when the User 200 activates Patch 110 through the use of a button, a sensor, a switch, or similar indicator on Patch 110.

An example, the User 200 interacts with Patch 110 to report progress in the treatment of the headache, such as indicating level of pain, level of relief.

The User 200 interaction with Patch 110 may be through wireless means such as from a smart phone or remote device.

An example, feedback from Patch 110 changes the stimulation protocol, such as adjusting one or more of intensity, signal waveform, and duration, to optimize efficacy and use of the electrical energy in Patch 110.

### Data Collection

An example, analysis of measurements from one or both of the Smart Controller 120 and Patch 110 may be performed by processing in a remote server, in the cloud, or on a computer separate from the Smart Controller but local to the User, such as a personal computer. The system analyzes this data and determines the most effective times to start and end each treatment protocol to optimize relief of headache.

An example, the Headache Detection and Treatment System 102 uses AI techniques such as pattern recognition and correlation analysis to correlate real-time data recordings of the User with larger population databases to produce comparative or predictive analyses. An example, machine-learning algorithms are employed to build up the User's headache history and provide specific predictors of headache activity.

An example, the time-based records of tissues are supplemented with data entered manually by one or more of the User and observers. The data recorded in the time-based database is sent to the cloud through a local network, such as a home mesh network, or directly over the Internet. The convenient and comfortable use of the Headache Detection and Treatment System 102 by the User 200 allows the system to collect data over a longer period of time without undue interference with lifestyle compared to conventional approaches to headache relief.

### Stimulation Protocol for Headache Treatment

In examples, user 200 selects a protocol of electrical stimulation, to be applied by patch 110 to the targeted nerve or nerves. The stimulation protocol may be automatically adjusted based on sensed parameters, adjusted by user 200 as the relief of headache progresses, or as directed by a medical professional.

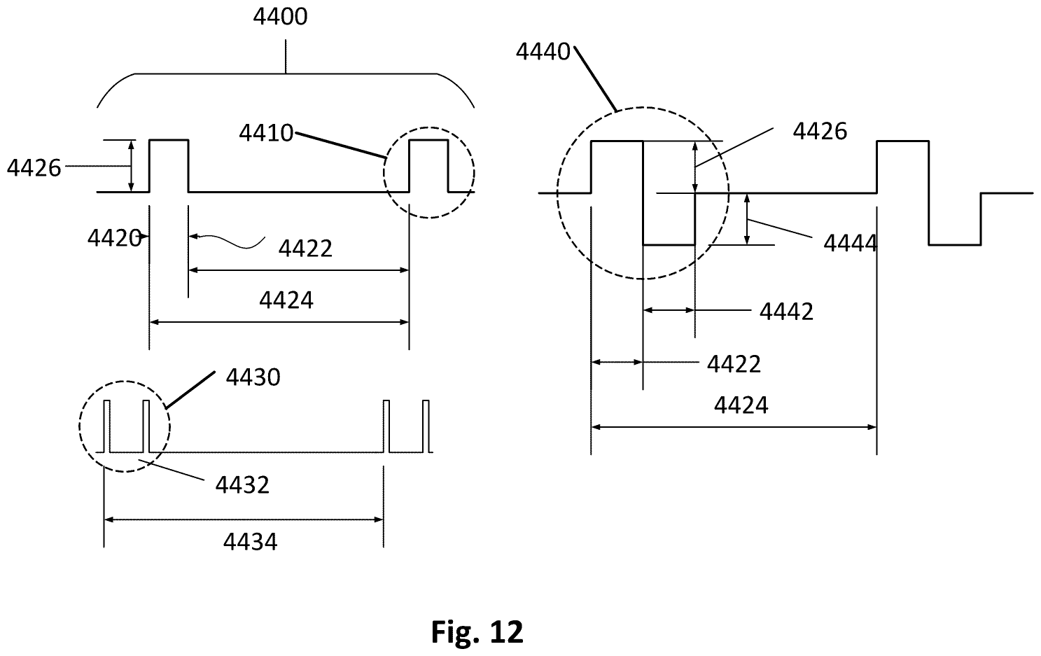

FIG. 12 illustrates example stimulation waveforms for treating headaches in accordance with example inventions. Patch 110 stimulates the tissue using a series of electrical pulses in a pattern of pulse sequence 4400 with a specific frequency, waveform, intensity and duration. Pulses 4410 may be applied at an intensity below that level which stimulates a painful sensation and below that level which wakes a user if the treatment occurs during sleep.

In examples, each pulse has a pulse high time 4420, a pulse low time 4422, a pulse period 4424, and a pulse amplitude 4426. Pulses 4410 may be monopolar pulses or bipolar pulses 4440. For monopolar pulses, the pulse period 4424 is the sum of the pulse high time 4420 and the pulse low time 4422. Bipolar pulses have a negative pulse width 4442 and a negative pulse amplitude 4444, for the purpose of balancing the DC bias of the sequence of stimulation pulses, and for the purpose of balancing for zero net energy into the tissues. The negative pulse width may differ from the pulse high time. The negative pulse amplitude may differ from the pulse amplitude. Pulse shapes are affected by the impedance coupling to the user's tissues and by the patch 110 output impedance, internal drive strength, and other factors, such that the pulses, whether monopolar or bipolar, may not be square waves.

One or more of the pulse high time 4420, the pulse low time 4422, the pulse period 4424, and the pulse amplitude 4426 may be adjusted. For bipolar pulses 4440, the negative pulse width 4442 and negative pulse amplitude 4444 may be adjusted from one user to another user, or from one application of a device to another on the same user. The pulse pattern may be adjusted during the course of a treatment.

Pulses may be output in bursts 4430. Each burst has two or more pulses 4410, or bipolar pulses 4440. Each burst has a burst pulse count 4432 and a burst period 4434. One or both of the burst pulse count and the burst period are adjustable for each user, or from one application of a device to another on the same user. The pulse frequency is the inverse of the pulse period. The burst frequency is the inverse of the burst period. Pulses or bursts may be adjusted by each user each time a patch 110 is applied, since effective intensity may be different according to skin condition, dampness, dryness, weight change, specific location of placement and other factors. In this manner, the electrical pattern of stimulation pulses is adjusted for each application/treatment.

In an example, the polarity of the electrical stimulation may be reversed during the course of a headache treatment. In examples, the pulses within one burst may all be of equal width. In examples, the pulses within one burst may be of varying widths, the width adjusted to optimize the stimulation for effectiveness.

In an example, the applied frequency of the stimulation pulses 4410 is in the range of 2 Hz to 150 Hz, and the current applied is up to 10 milliamps. In an example, pulses 4410 singly or in bursts 4430 have pulse high times 4420 in the range of 100 to 500 microseconds, and pulse low times 4422 in the range of 100 to 500 microseconds, and with burst frequency and pulse frequency for single pulses in the range of 2 Hz to 50 Hz, and the current applied is up to 10 milliamps.

In examples, the pulses within one burst may be evenly spaced and/or are all of equal width. In examples, the pulses within one burst may be unevenly spaced and/or have varying widths, with the width adjusted to optimize the stimulation for effectiveness. In examples, the pulses within one burst may have consistent amplitude. In examples, the pulses within one burst may have unequal amplitudes.

In an example, one or more of pulse rise time, pulse fall time, pulse overshoot, and pulse undershoot are adjusted by one or both of patch 110 and smart controller 140. Changes in pulse shape, including one or more of rise time, fall time, overshoot and undershoot, allow the patch to optimize use of power during the application of a treatment protocol. Optimizing the power used in a treatment allows a patch with a given design to apply more stimulation when compared to a patch without the means to optimize power delivery. Pulse shape is limited by one or both of patch and smart controller such that delivered energy, rate of energy delivery, magnitude of currents and/or voltages all meet the requirements for effective nerve stimulation at the applied position.

In an example, smart controller 140 adjusts the intensity of applied pulses, or the duration of application, or both, using data exchanged with patch 110 and its processor 118. The exchanged data includes data from the monitoring device or devices, described below included in headache treatment system 102.

In an example, one or both of patch 110 and smart controller 140 adjusts the intensity or the duration of the applied pulses, or both. In an example, user 900 adjusts the intensity and the duration of the applied pulses, or both. All adjustments may be limited to preset ranges.

In an example, one or both of patch 110 and the smart controller 140 operate to select one of a variety of hardware configurations, each hardware configuration on the patch specified to limit one or more of pulse rise time, pulse fall time, pulse overshoot, and pulse undershoot. One example uses a bank of capacitors, switched into the pulse application circuit under control of the patch, to optimize the load and its effect on the driven pulse voltage and current. A second example uses a bank of inductive loads. A third example uses a bank of resistive loads.

As disclosed, embodiments treat headaches by stimulating the nerves with transcutaneous electrical stimulation. In contrast, known solutions of subdural electrical treatment for headaches involves significant expense, and risks related to surgical procedures and the implanted device.

Several examples are specifically illustrated and/or described herein. However, it will be appreciated that modifications and variations of the disclosed examples are covered by the above teachings and within the purview of the appended claims without departing from the spirit and intended scope of the invention.

What is claimed is:

1. A method of treating a headache, the method comprising:
affixing a patch externally on a dermis of a user on a head of the user adjacent to one or more targeted nerves, the patch comprising a flexible substrate, a processor directly coupled to the substrate, and electrodes directly coupled to the substrate; and
activating the patch to initiate a treatment session, the activating comprising generating electrical stimuli to the targeted nerves via the electrodes.

2. The method claim 1, the patch affixed on a forehead of the user, the one or more targeted nerves comprises bilateral supraorbital nerves or supratrochlear nerves.

3. The method claim 1, the patch affixed on a back of the head, the one or more targeted nerves comprises bilateral occipital nerves.

4. The method of claim 1, the patch comprising one or more sensors directly coupled to the substrate, the sensors detecting a degree of the headache.

5. The method of claim 4, the sensors sensing one or more of chemical, thermal, electrical field, magnetic field, audio or motion.

6. The method of claim 5, further comprising adjusting the treatment session in response the sensing.

7. The method of claim 6, the electrical stimuli comprising a series of pulses with a pattern comprising an intensity and a duration, the adjusting comprising adjusting the intensity or the duration of the pattern.

8. The method of claim 1, the electrical stimuli having a frequency comprising 1 Hz to 100 Hz, an amplitude comprising 10 to 50 milliamps, a duration comprising 1 minute to 1 hour and a pulse width comprising 25 to 500 microseconds.

9. The method of claim 5, further comprising:
determining a target charge level for the treatment based at least in part on the sensing by the sensors;
outputting a series of pulses from the electrodes;
for each pulse outputted, measuring a charge value of the pulse and compare the charge value to the target charge level;
if the charge value is greater than the target charge level, reducing a strength level of a subsequent outputted pulse;
if the charge value is less than the target charge level, increasing the strength level of a subsequent outputted pulse; and
repeating the determining, outputting and measuring.

10. The method of claim 9 the determining the target charge level Q_{target} comprises generating an acquisition series of pulses and
$${Q}_{target} = {\sum }_{i = 1}^{T * f} {Q}_{pulse} ( i ) ,$$
where T is a duration of the acquisition series of pulses, f is a count of pulses for one treatment acquisition series of pulses and Qₚᵤₗₛₑ (i) is a measured charge of each of the acquisition series of pulses.

11. The method of claim 9, the patch further comprising electronic circuitry directly coupled to the substrate and comprising a differential integrator, the charge value of the pulse based on an output of the differential integrator.

12. A headache treatment system comprising:
a patch adapted to be externally affixed on a dermis of a user on a head of the user adjacent to one or more targeted nerves, the patch comprising a flexible substrate, a processor directly coupled to the substrate, and electrodes directly coupled to the substrate; and
the processor adapted to activate the patch to initiate a treatment session, the activating comprising generating electrical stimuli to the targeted nerves via the electrodes.

13. The headache treatment system claim 12, the patch adapted to be affixed on a forehead of the user, the one or more targeted nerves comprises bilateral supraorbital nerves or supratrochlear nerves.

14. The headache treatment system claim 12, the patch adapted to be affixed on a back of the head, the one or more targeted nerves comprises bilateral occipital nerves.

15. The headache treatment system claim 12, the patch comprising one or more sensors directly coupled to the substrate, the sensors detecting a degree of the headache.

16. The headache treatment system claim 15, the sensors sensing one or more of chemical, thermal, electrical field, magnetic field, audio or motion.

17. The headache treatment system claim 16, the processor further adapted to adjusting the treatment session in response the sensing.

18. The headache treatment system claim 17, the electrical stimuli comprising a series of pulses with a pattern comprising an intensity and a duration, the adjusting comprising adjusting the intensity or the duration of the pattern.

19. The headache treatment system claim 12, the electrical stimuli having a frequency comprising 1 Hz to 100 Hz, an amplitude comprising 10 to 50 milliamps, a duration comprising 1 minute to 1 hour and a pulse width comprising 25 to 500 microseconds.

20. The headache treatment system claim 16, the processor further adapted to:
determining a target charge level for the treatment based at least in part on the sensing by the sensors;
outputting a series of pulses from the electrodes;
for each pulse outputted, measuring a charge value of the pulse and compare the charge value to the target charge level;
if the charge value is greater than the target charge level, reducing a strength level of a subsequent outputted pulse;
if the charge value is less than the target charge level, increasing the strength level of a subsequent outputted pulse; and
repeating the determining, outputting and measuring.
